(19) United States
(12) Patent Application Publication
(10) Pub. No.: US 2004/0236191 A1
(43) Pub. Date: Nov. 25, 2004

(54) System and method for identifying and labeling livestock products, and managing data associated with those products

(76) Inventors: Steven A. Poliska, Rockaway, NJ (US); Daniel V. Barus, North Hanover, NJ (US); Richard L. Sibbel, Ankeny, IA (US)

Correspondence Address: LARRY LIBERCHUK LLC, PO BOX 385, EDGEWATER, NJ, 07020, US

(21) Appl. No.: 10/847,985

(22) Filed: May 17, 2004

Related U.S. Application Data
(60) Provisional application No. 60/471,874, filed on May 19, 2003.

(51) Int. Cl. A61B 5/00

(52) U.S. Cl. 600/300

(57) ABSTRACT

A system and method identify and label livestock products with such information as country, place and date of origin, among other things. In the inventive system and method, a tag is provided with a unique identifier. The tag is inserted into a predetermined meat portion of an animal carcass. The tag remains inserted while the animal carcass is processed. The tag is removed prior to packaging the predetermined meat portion, and a label with another identifier is attached onto the packaged meat portion. The two identifiers are associated with each other, so that the label provides identifying information about the livestock product.

### CROSS-REFERENCE TO RELATED APPLICATIONS

This application claims the benefit of U.S. Provisional Application No. 60/471,874, filed May 19, 2003.

### BACKGROUND OF THE INVENTION

The present invention relates to a system and method for identifying and labeling livestock products during a fabrication process, and managing data associated with those products and, more particularly, to identification/temperature-sensing tags and bar-code labels affixed to animal carcasses, a message panel for data collection and verification, a data warehousing and management system located on a local, wide area or global computer network such as the Internet, and a web site, the so-called portal, for entering and displaying animal-related information to registered users.

The cattle industry, being a representative example of the livestock industry, comprises several vertically integrated segments, such as producers, feedlots, packing plants, and wholesalers/retailers. The cattle industry cycle starts with the commercial cattle producers maintaining herds of cows for producing calves. The calves are grown to a certain size and then moved to feedlots, where tens of thousands of head of cattle or other ruminants are cared for in various stages of growth. In the feedlots, the ruminants are fed a special diet to reach their optimum weight and size while trying to keep the animals healthy. Subsequently, the animals are sent to the packing plants for slaughter. Also in the packing plants the animal carcasses are cut into various portions or cuts of meat. These cuts of meat are packed, chilled and shipped by the packers to the wholesalers and/or retailers for distribution to the public.

The present invention is directed to a slaughter facility phase. Wholesalers, retailers and consumers express many concerns, for various reasons, about the identification of origin and tracking of meat products and allowing important identification information, such as country, place and date of origin, to propagate to those levels.

A need, therefore, exists for a system and method for efficiently, safely and accurately identifying and tracking various meat products at a slaughter facility, for providing this information to wholesalers, retailers, consumers, and for optimally storing and managing the accumulated data in order to devise the informed decision-making strategies.

### SUMMARY OF THE INVENTION

It is an object of the present invention to provide a system and method for identifying and labeling livestock products.

The above and other objects are achieved by such inventive system and method. According to one embodiment of the present invention, a tag is provided with a unique identifier. The tag is inserted into a predetermined meat portion of an animal carcass. The tag remains inserted while the animal carcass is processed. The tag is removed prior to packaging the predetermined meat portion, and a label with the identifier is attached onto the packaged meat portion. The label provides identifying information about the livestock product.

### BRIEF DESCRIPTION OF THE DRAWINGS

The foregoing and other features of the present invention will be more readily apparent from the following detailed description and drawings of one or more illustrative embodiments of the present invention in which:

FIG. 1 is a general flow diagram of animal processing at a slaughter facility;

FIG. 2A is a more detailed flow diagram of animal processing at a slaughter facility;

FIG. 2B is a continuation of animal processing flow diagram of FIG. 2A;

FIG. 3 shows various designs of tags;

FIG. 4 illustrates a representative placement of a tag at a tag insertion station and tag tracing during the processing cycle;

FIG. 5 illustrates a representative message panel with information filled at a tag insertion station and a representative insertion of the tag into a carcass;

FIG. 6 illustrates tag repositioning prior to cutting through a big loin section;

FIG. 7 illustrates representative cutting and trimming of various meat sections;

FIG. 8 illustrates representative cutting and trimming of various meat sections and grouping of those sections for identification;

FIG. 9 illustrates bar-code labels attached to the meat products;

FIG. 10 illustrates the system and process of obtaining information from RFID readers and tags and communicating the information to a back-end server and database for storage and management.

### DETAILED DESCRIPTION OF PREFERRED EMBODIMENTS

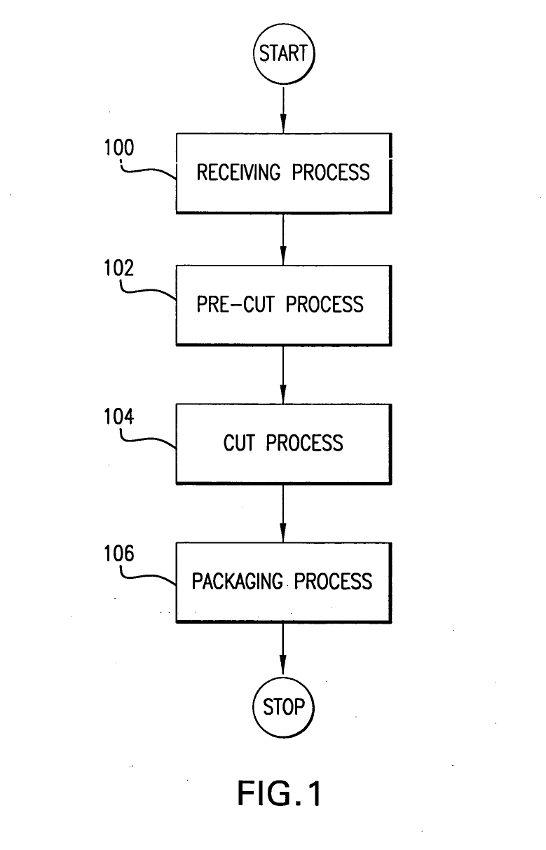

The present invention applies to livestock industry in general. One representative example of the present invention is directed to a pork industry. To facilitate the description of the present invention its embodiments are presented herein in connection with pork production and processing at a slaughter facility, also known as a packer. FIG. 1 is a general flow diagram of animal processing at a slaughter facility or packer. According to the figure, in step 100 pigs are typically delivered by producers or feedlot operators, etc., to a packer's receiving dock. Then, the pre-cut process is carried out in step 102, followed by a cut process to.produce various meat sections or cuts in step 104. Finally, the meat cuts are packaged for delivery to wholesalers, retailers, etc., in step 106.

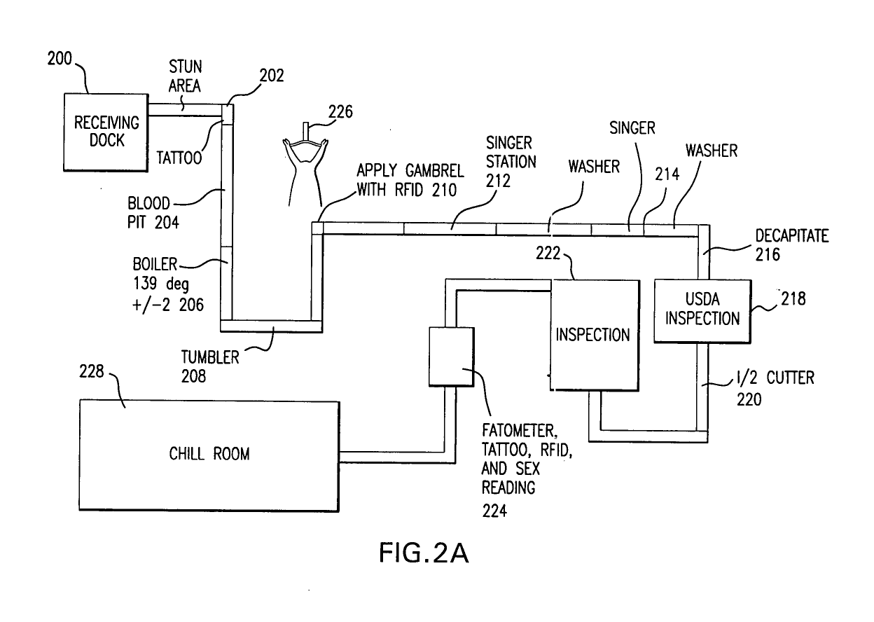

FIGS. 2A and 2B are more detailed flow diagrams of animal processing at the slaughter facility. After the arrival to a receiving dock in step 200, pigs are stunned and individually tattooed with a particular identification number, which may uniquely represent each animal or a group of animals, in step 202. Then, blood is drained from the slaughtered pigs at a blood pit in step 204, and the pigs are boiled at a boiler station at a particular temperature in step 206. A representative boiling temperature is in the range of 137 to 141 degrees Fahrenheit.

After some additional processing, such as tumbling in step 208, a gambrel 226, which is a frame for hanging a carcass by the legs, is applied to each individual pig in a trolley tracking system, as shown in step 210 of FIG. 2A. The trolley tracking system, among other things, facilitates moving the carcasses efficiently from one processing stage to another along the cycle and from one processing station to another. Each gambrel 226 may or may not include a Radio Frequency ID (RFID) device attached thereto in order to uniquely identify each carcass or a group of carcasses.

As further shown in FIG. 2A, the carcasses are moved from one processing station to another on gambrels. The representative processing cycles include singing in step 212, washing in step 214, decapitating in step 216. After the USDA inspection station in step 218, the carcasses are substantially cut in half by a half-cutting machine in step 220. Further inspection follows the half cutting in step 222, and the carcasses then arrive, still suspended on the gambrels, at a station for tattoo reading, sex reading, fat reading, etc., as shown in step 224 of FIG. 2A.

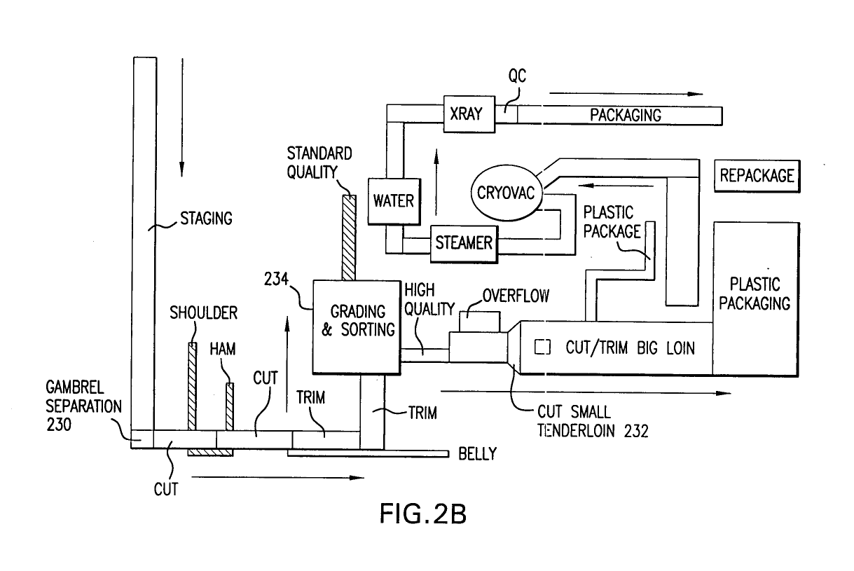

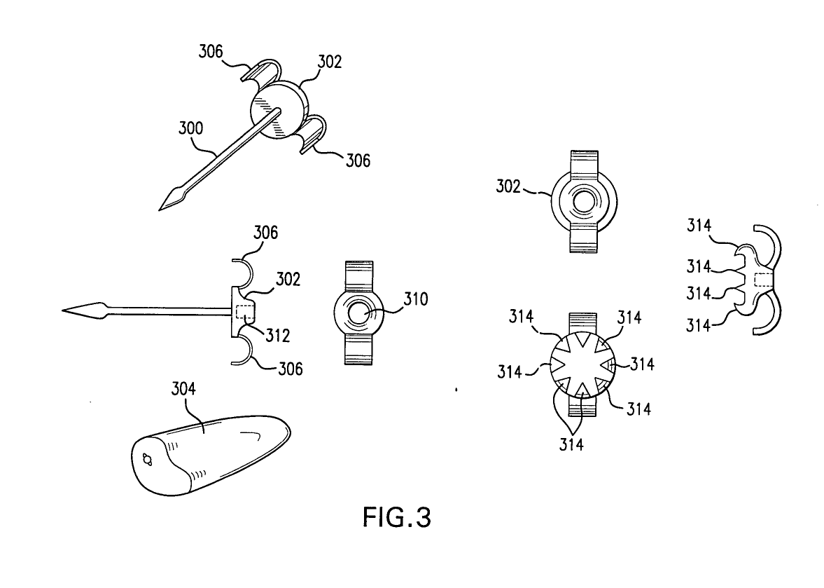

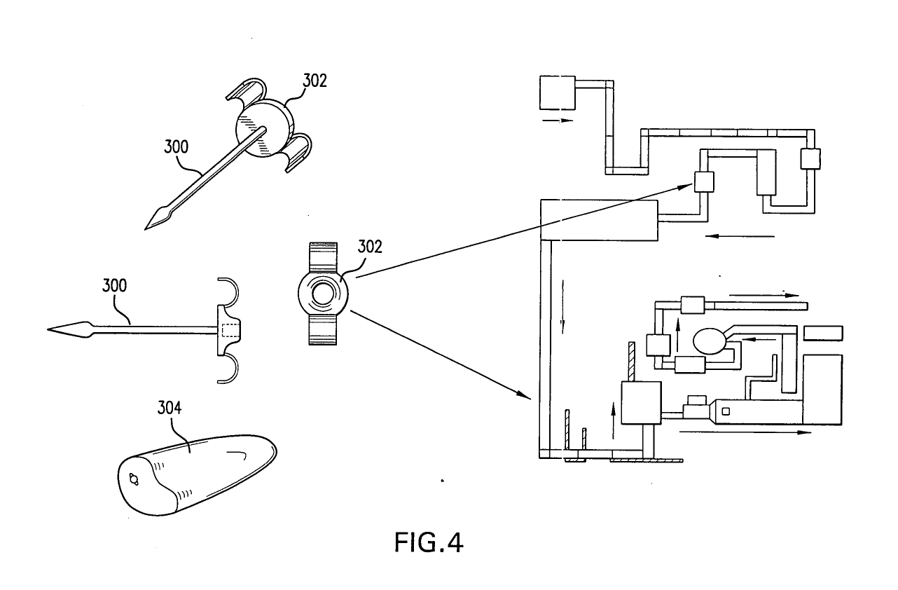

As one representative example according to an embodiment of the present invention, at this stage of the processing cycle FIG. 5 shows a temporary identification tag 500 is inserted in a section 502 of the animal after the carcass 504 has been substantially cut in half. FIG. 5 illustrates a representative insertion of the tag 500 into the carcass 504. The temporary ID tag 500 may be a sharp elongated pin-like device 300 for penetrating into the carcass meat portion 304 and having a top section 302, similar to a nail head, with one or more flanges 306 for ease of grasping the tag 500 by an operator 308 and inserting and/or removing it from the carcass 504, as shown in FIGS. 3 and 4. The top section 302 has an internal chamber 310 fully protected and sealed from an external environment. The chamber 310 is used for storing an electronic passive device 312, such as a digital Integrated Circuit (IC), capable of storing data and transmitting the stored data via a wireless medium in response to a request from a Radio Frequency (RF) reader.

The tag design, of course, is not limited to the one described above. For example, instead of the elongated pin-like body, the tag may include a top section, as described above, and a number of short protruding hooks 314 capable of being secured to the carcass surface without penetrating the meat portion, as shown in the right portion of FIG. 3. Other designs may be used that fulfill the above functions of the tag, such as being securely embedded into the carcass meat portion and containing an electronic device for data storage and transmission.

The temporary tag is preferably inserted into the rib section 502 of the animal as representatively shown in FIG. 5. The rib section 502 is the preferred placement for the tag, because the rib section can be easily accessed by an operator after the carcass has been substantially cut in half.

Preferably prior to inserting the tag into the carcass, the operator scans the tag with an RFID reader. The RFID reader may be a hand-held portable device or alternatively a stationary device. Once the tag is read by the RFID reader, the information stored in the tag is transferred via a wireless medium to the RFID reader, which is connected to a server via a wireless or wired connection. One or more application programs running on the server create a record for each carcass. As shown in FIG. 5, the record is then displayed on a message panel 506 at the operator station where the tag insertion has taken place. Once the record fields are populated, the operator may enter new information, modify the existing one, etc. As further shown in FIG. 5, the “Release” button is provided on the message panel 506 for signaling the record completion task.

After this data collection station, the carcasses are chilled to a pre-determined temperature at a chill room in step 228, as illustrated in FIG. 2A. After the chill room, additional processing takes place as shown in FIG. 2B, the carcasses are separated from the gambrels in step 230 to allow for manual cutting and trimming operations. Namely, shoulder and other parts are cut from the carcass that up until this point has been substantially cut in half.

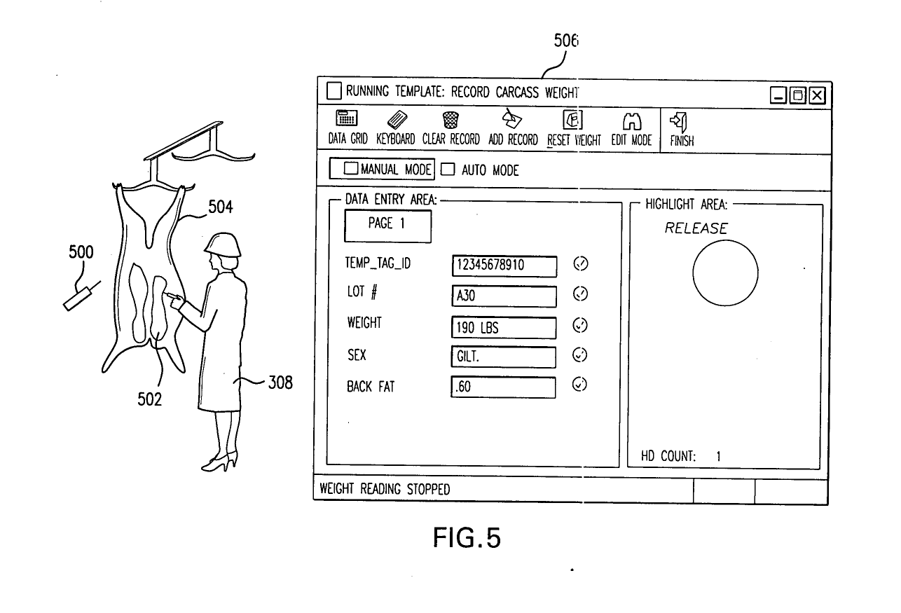

As shown in more detail in FIG. 6, after the gambrel separation, the carcass is further cut to expose side meat portions. The tag 500 is then manually repositioned in step 600 from the top to the side portion of the carcass once the sides become easily accessible following the cutting stages of the processing. The tag repositioning is required to avoid interference with one or more saws 602 for cutting through the big loin portion 304 of the animal as illustrated in FIG. 6. The repositioned tag thus remains inserted in the big loin.

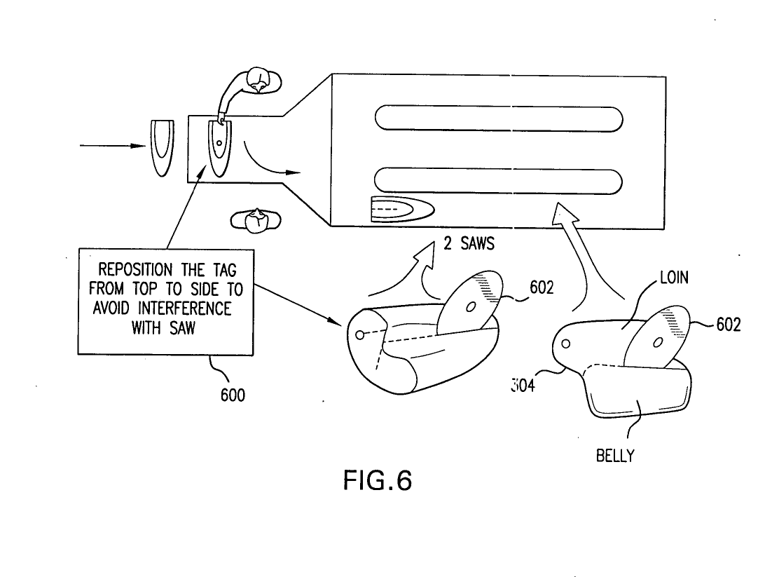

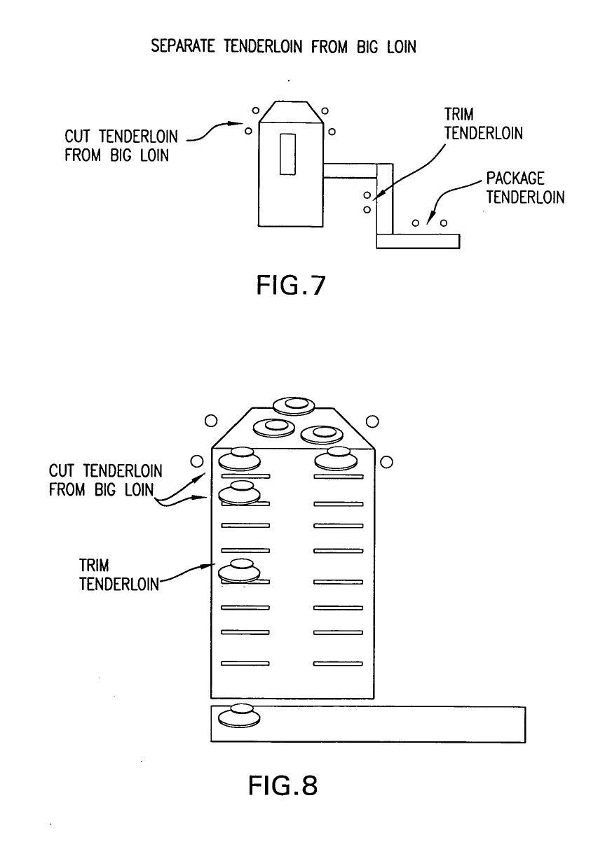

Returning to FIG. 2B, after additional processing of cutting, trimming, grading and sorting in step 234, a tenderloin is cut from the big loin in step 232 (also shown in FIG. 7). These two meat portions, however, are kept together in the same area or bin so that the tenderloin can be easily identified on the basis of the tag that remains inserted in the big loin as illustrated in FIG. 7. The tenderloin and big loin continue moving in tandem through additional stages of the processing cycle where the tenderloin is fuirther trimmed by an operator in step 800 as shown in FIGS. 7 and 8.

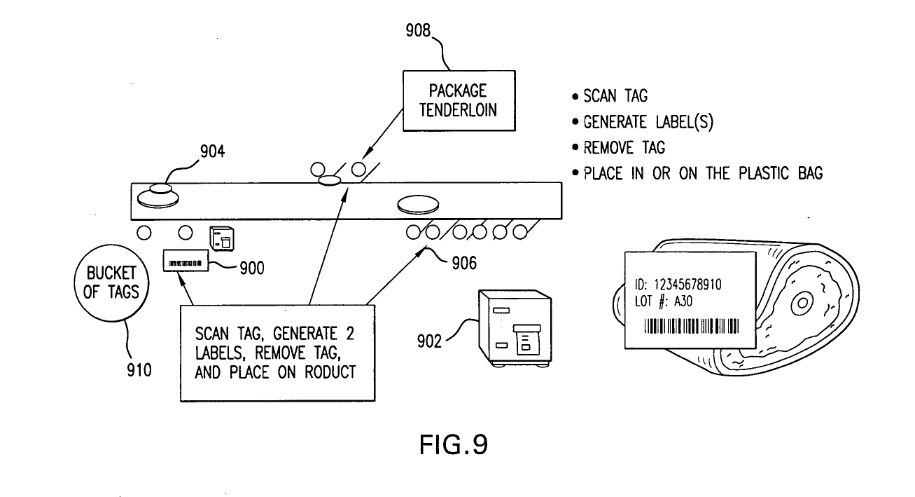

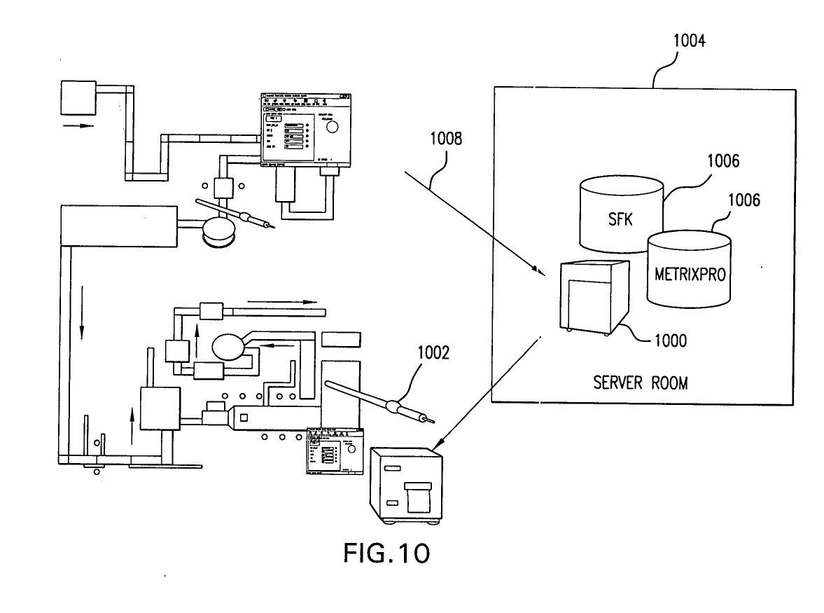

FIG. 9 illustrates various operations at the end of the trimming stage. The tag is read by the RFID reader. In particular, the tag emits an RF signal that uniquely identifies the particular meat portion (tenderloin and big loin) when energized by the RFID reader. A number of pre-printed bar-code labels 900, arranged in pairs such that the first bar-code label is identical to the second bar-code label in any given pair, are available to operators for packaging the meat product. Each pair of the bar-code labels, printed in step 902, corresponds to a tag. The cross-reference of each tag with a pair of labels is maintained on a server 1000 (FIG. 10), such that the information in each tag is contained in the bar-code label. When the tag is removed from the big loin in step 904, one bar-code label is placed on a plastic bag for packaging the big loin in step 906 and another bar-code label is placed on a plastic bag for packaging the tenderloin in step 908, as shown in FIG. 9. At the end of cycle, the RF tags are collected and washed in step 910 so that they can be re-used on other carcasses.

FIG. 10 illustrates the system and process of obtaining information via RFID ddreaders and tags in step 1002 and communicating the information in step 1008 to a back-end server and database for storage and management. The information on animal's place and country of origin, for example, as well as other data, is forwarded to a remotely located back-end portal 1004. The portal comprises server 1000 and database 1006 for document management, statistical processing of information, and report generation.

Subscribers to the system can use the Internet or other network to access the data in the database 1006 and to get the system to run individualized reports for them. The report creation software at the portal server allows subscribers to query the database as desired.

The main portal page for the website has links to other web pages and includes application programs that may be executed by clicking on various screen “buttons” or “tabs”. Some of the representative pages are:

HOME Main Web Page (home page)

Data Mgmt—Web Page for uploading data

Admin—Web Page for managing passwords and users in organization

TASK—Web Page for managing and querying tasks specific to the development of MyGAM and

BeefMetrix (GAM use only)

A security protocol is provided for the web site. In particular, by logging into the system, the subscriber's profile is being checked for authentication. Further, all applications that run in the portal support “single sign-on”. This means that the subscriber signs into the portal one time, and can navigate to all linked applications without having to manually sign onto them.

In another embodiment of the present invention, a temperature sensor is incorporated into the tag. Hanging meat, once it has undergone the initial processing, must be chilled to near freezing before continuing fabrication in the meat manufacturing plant. The amount of chilling that is required to fully chill the hanging carcass in the chill room has been determined by engineering formulas and air movement strategies in the coolers. Validation of core temperatures—chilled to correct temperature—in the hanging carcass has never been a standard Quality Control procedure in packing plants.

According to this embodiment, a temporary RF tag includes a temperature sensor to validate core temperatures in the deep meat muscles. This device allows for the simultaneous delivery of individual identification and temperature sensing capacity utilizing a single RFID reader. This quality control validation of core muscle temperature ensures the correct chilling of meat to its core and raises the product safety value as a food source.

It will be appreciated that tags other than RF-based may also be used in the embodiments of the present invention. Various marking codes, such as Data Matrix or bumpy bar codes, may be used on the tags in conjunction with the corresponding readers. The marking codes embedded into the tags may be 2 or 3-dimensional, as known to those skilled in the art.

While the present invention has been described and illustrated in connection with the above embodiments, many variations and modifications, as will be evident to those skilled in the art, may be made without departing from the spirit and scope of the present invention. The present invention is thus not to be limited to the precise details of methodology or construction set forth above, as such variations and modifications are intended to be included within the scope of the present invention.

What is claimed is:

1. A method for identifying a livestock product, comprising:
providing a tag with a first identifier;
inserting said tag into a predetermined meat portion of an animal carcass;
processing said animal carcass while said tag remains inserted into said predetermined meat portion;
removing said tag prior to packaging said predetermined meat portion; and
attaching a label with a second identifier onto the packaged meat portion, said second identifier associated with said first identifier such that said label provides identifying information about said livestock product.

2. The method according to claim 1, wherein said first and second identifiers are the same.

3. The method according to claim 1, further comprising cutting said animal carcass prior to inserting said tag in order to expose said predetermined meat portion.

4. The method according to claim 3, wherein said animal carcass is substantially cut into 2 halves.

5. The method according to claim 1, wherein said processing includes further cutting, trimming, grading and sorting.

6. The method according to claim 5, further comprising repositioning said tag during said processing in order to avoid interfering with automatic cutting tools.

7. The method according to claim 1, wherein said tag includes a device for transmitting data containing said first identifier over radio frequencies.

8. The method according to claim 1, wherein said first identifier is a 2-dimensional marking embedded in said tag.

9. The method according to claim 1, wherein said first identifier is a 3-dimensional marking embedded in said tag.

10. The method according to claim 1, wherein said label is bar code readable.

11. The method according to claim 1, wherein said tag comprises a sensor for measuring chilling temperature of said animal carcass during said processing.

12. The method according to claim 1, wherein said first and second identifiers are conveyed, as computer-readable data, to a database server in order to populate a database with information about livestock products.

13. A system for identifying a livestock product, comprising:
a tag with a first identifier for inserting into a predetermined meat portion of an animal carcass, said tag remaining inserted into said predetermined meat portion while said animal carcass is processed, said tag being removed prior to packaging said predetermined meat portion;
a label with a second identifier for attaching onto the packaged meat portion, said second identifier associated with said first identifier such that said label provides identifying information about said livestock product; and
a database server for receiving said first and second identifiers, as computer-readable data, in order to populate a database with information about livestock products.

14. The system according to claim 13. wherein said tag includes a device for transmitting data containing said first identifier over radio frequencies.

15. The system according to claim 13, wherein said first identifier is a 2-dimensional marking embedded in said tag.

16. The system according to claim 13, wherein said first identifier is a 3-dimensional marking embedded in said tag.

17. The system according to claim 13, wherein said label is bar code readable.

18. The system according to claim 13, wherein said tag comprises a sensor for measuring chilling temperature of said animal carcass during said processing.

19. The system according to claim 13, wherein said first and second identifiers are the same.
